(19) United States
(12) Patent Application Publication
(10) Pub. No.: US 2023/0414262 A1
(43) Pub. Date: Dec. 28, 2023

(54) HIGH-STRENGTH ABSORBABLE COMPOSITE ACTIVE INTERNAL FIXATION DEVICE AND PREPARATION METHOD THEREFOR

(71) Applicant: EAST CHINA UNIVERSITY OF SCIENCE AND TECHNOLOGY, Shanghai (CN)

(72) Inventors: Changsheng LIU, Shanghai (CN); Yulin LI, Shanghai (CN); Wei LUO, Shanghai (CN); Can CHEN, Shanghai (CN)

(21) Appl. No.: 17/905,561

(22) PCT Filed: Mar. 2, 2021
(86) PCT No.: PCT/CN2021/078708

§ 371 Date: Jul. 13, 2023

(30) Foreign Application Priority Data
Mar. 2, 2020 (CN) 202010135441.8

(51) Int. Cl. A61B 17/86; A61L 27/12; A61L 27/18; A61L 27/54; A61L 27/58; A61L 31/12; B29B 9/12; B29C 71/02; B29C 45/77; B29C 45/78; B29C 48/92

(52) U.S. Cl. CPC A61B 17/86; A61L 27/12; A61L 27/18; A61L 27/54; A61L 27/58; A61L 31/127; B29B 9/12; B29C 71/02; B29C 45/77; B29C 45/78; B29C 48/92; B29K 2067/046

(57) ABSTRACT

The present invention relates to a high-strength absorbable composite active internal fixation device and a preparation method therefor. Specifically, the present invention discloses the internal fixation device and its preparation method, which possess excellent biological activity and mechanical properties.

### TECHNICAL FIELD

The present invention relates to the field of biomedical instruments, and particularly to a high-strength absorbable composite active internal fixation device and a preparation method therefor.

### BACKGROUND

Bone injury is a disease which occurs widely in clinic with a high probability of occurrence, and internal fixation is the most common treatment means. Traditional metal internal fixation materials must be removed by a second operation after healing, which increases the pain and economic burden of patients. Using bioabsorbable materials instead of traditional non-absorbable materials has become a development trend. Although such products are already available, the poor mechanical properties and inflammatory reaction thereof often lead to implant failures.

Polylactic acid is a degradable polymer material which is safe to human body and friendly to environment, and can be used for preparing surgical sutures, injection microcapsules, degradable bone nails, implants, artificial bones, artificial skin, etc. Polylactic acid is characterized in that metabolites thereof are non-toxic and are eventually converted into carbon dioxide and water in the body.

The existing absorbable internal fixation implant products, represented by polylactic acid, generally have the problems of low strength and easy fracture, which are the main reasons for implant failures. Among which, mechanical decay of materials caused during machining is an unsolved problem so far.

### SUMMARY

The purpose of the present invention is to provide an internal fixation device with excellent biological activity and mechanical properties and a preparation method therefor.

In a first aspect of the present invention, a preparation method for an internal fixation device is provided, comprising the following steps:
1) Providing a first mixture, wherein the first mixture contains bioactive nanoparticles, a catalyst and biomedical grade monomers;
2) Conducting polyreaction to the first mixture to obtain a first polymer;
3) Conducting granulation to the first polymer;
4) Injecting a granulation product into an injection molding machine to form a first blank by injection molding;
5) Annealing the first blank to obtain a second blank;
6) Conducting extrusion molding to the second blank, and conducting precision machining to the product obtained to prepare an internal fixation device.

In another preferred embodiment, the bioactive nanoparticles are inorganic particles selected from the following group: hydroxyapatite, tricalcium phosphate, calcium sulphate, calcium phosphate, magnesium sulphate, or a combination thereof.

In another preferred embodiment, the inorganic particles are surface modified inorganic particles, and surface modification is conducted by a modified material selected from the following group: silane coupler, polylactic acid (such as low molecular weight polylactic acid), polylactic acid-polycaprolactone copolymer, or a combination thereof.

In another preferred embodiment, particle diameter of the inorganic particles is 5-30000 nm, preferably 10-25000 nm, preferably 20-20000 nm, preferably 30-500 nm, preferably 40-400 nm, and more preferably 50-350 nm.

In another preferred embodiment, mass content of the modified material in the surface modified inorganic particles is 0.001-40 wt %, preferably 0.01-20 wt %, and more preferably 0.015-20 wt %.

In another preferred embodiment, the catalyst is selected from the following group: zinc oxide, stannous octoate, stannous chloride, butyl magnesium, or a combination thereof.

In another preferred embodiment, the biomedical grade monomers are selected from the following group: L-lactide, D(+)-lactide, DL-lactide, trimethylene carbonate, caprolactone, glycolide, or a combination thereof.

In another preferred embodiment, mass content of the bioactive nanoparticles in the first mixture is 0.001-80 wt %, preferably 0.01-60 wt %, and more preferably 0.01-40 wt %.

In another preferred embodiment, mass content of the catalyst in the first mixture is preferably 0.03-0.80 wt %, and more preferably 0.04-0.60 wt %.

In another preferred embodiment, mass content of the biomedical grade monomers in the first mixture is 20-99.999 wt %, preferably 40-99.99 wt %, and more preferably 60-99.99 wt %.

In another preferred embodiment, number-average molar mass of the first polymer is 50-1000 kDa, preferably 80-800 kDa, and more preferably 100-600 kDa.

In another preferred embodiment, the first polymer is selected from the following group: poly-L-lactic acid homopolymer, poly(D,L-lactide) homopolymer, DL-lactide-caprolactone copolymer and L-lactide-glycolide copolymer.

In another preferred embodiment, the granulation has one or more characteristics selected from the following group:
1) The granulation is conducted using a single screw rod or double screw rods;
2) Granulation temperature is 40-300° C., preferably 60-260° C., and more preferably 100-240° C.;
3) Rotating speed of the single screw rod or double screw rods is 20-60 rpm, preferably 30-50 rpm, and more preferably 35-45 rpm.

In another preferred embodiment, the injection molding has one or more characteristics selected from the following group:
1) Bucket temperature of the injection molding machine is 50-250° C., preferably 80-220° C., and more preferably 120-200° C.;
2) Mold temperature is 10-150° C., preferably 15-120° C., and more preferably 20-80° C.;
3) Injection time is 2-30 s, and preferably 3-25 s;
4) Injection pressure is 0.2-20 MPa, and preferably 1-15 MPa;
5) Dwell time is 5-100 s, and preferably 10-60 s;
6) Dwell pressure is 1-20 MPa, and preferably 3-15 MPa.

In another preferred embodiment, the annealing has one or more characteristics selected from the following group:
1) The annealing is to anneal the first blank together with a mold;
2) Cooling rate of the annealing is 5-50° C./min, and preferably 10-40° C./min;
3) Cooling time of the annealing is 4-15 min, and preferably 5-10 min.

In another preferred embodiment, the extrusion molding has one or more characteristics selected from the following group:
1) Extrusion temperature of the extrusion molding is 60-200° C., preferably 80-180° C., and more preferably 100-160° C.;
2) Extrusion pressure of the extrusion molding is 50 N-100 kN, and preferably 100 N-50 kN.

In another preferred embodiment, the extrusion molding is repeated in step 6).

In another preferred embodiment, the extrusion molding is repeated for 2-10 times, and preferably for 3-6 times.

In a second aspect of the present invention, an internal fixation device is provided, wherein the internal fixation device is prepared by the method of the first aspect of the present invention.

In another preferred embodiment, the internal fixation device has one or more characteristics selected from the following group:
1) Flexural strength of the internal fixation device is 100-300 MPa, preferably 150-300 MPa, and more preferably 250-300 MPa;
2) Flexural modulus of the internal fixation device is 2.5-5 GPa, and preferably 3-4 GPa.

In another preferred embodiment, the material forming the internal fixation device is selected from the following group: poly-L-lactic acid, poly(D,L-lactide), DL-lactide-caprolactone copolymer and L-lactide-glycolide copolymer.

In another preferred embodiment, the internal fixation device is selected from the following group: an internal fixation compression screw, an interference screw, a rib rod and an internal fixation plate.

It should be understood that each of the above technical characteristics of the present invention and each of the technical characteristics specifically described in the following (e.g., embodiments) may be combined with each other in the scope of the present invention, thereby constituting a new or preferred technical solution. Due to space limitations, it will not be repeated herein.

### DESCRIPTION OF DRAWINGS

FIG. 1 shows a picture of an internal fixation rod obtained in embodiment 1.

FIG. 2 shows mechanical test results of an internal fixation device.

FIG. 3 shows ALP staining results of embodiment 3.

FIG. 4 shows quantitative analysis results of ALP staining in embodiment 3.

FIG. 5 shows a picture of an internal fixation screw in embodiment 1 and an injection screw in reference example 1.

FIG. 6 shows mechanical comparison results between a screw obtained in embodiment 1 and an injection screw in reference example 1.

FIG. 7 is a micro-morphological picture of the surface and internal fracture surface of the internal fixation bar obtained in example 1.

### DETAILED DESCRIPTION

After long-term and in-depth research, and through an improved preparation process, the inventor has accidentally prepared an internal fixation device with excellent biological activity and mechanical properties. On this basis, the inventor completes the present invention.

### Internal Fixation Device

The present invention develops an active nanoparticle composite technique, and a low damage injection molding technique combined with cold extrusion secondary orientation and crystallization induced autofrettage technique, so as to significantly improve the biological activity and mechanical strength of an implant material, reduce the mechanical property degradation rate of a device in the early stage of implantation, attenuate inflammatory reaction, and improve the safety and effectivity of an absorbable internal fixation product for a user.

The biological activity includes but is not limited to an ability to promote cell adhesion, osteoinductivity and osteoconductibility.

The internal fixation device has the functions of in vivo degradation and absorption, and has osteoinductive activity.

The biological activity of the internal fixation device is manifested by that the material has good osteoconductibility and an ability to induce osteogenic regeneration, which can promote bone tissue regeneration in situ.

### Preparation Method

The present invention provides a preparation method for the internal fixation device, comprising the following steps:
1) Initiating polymerization of biomedical grade monomers by bioactive nanoparticles in situ;
2) Collecting a biomedical grade polylactic acid polymer obtained from the polymerization, and making the polymer into a blank by lossless machining;
3) Machining the blank into a section bar by the autofrettage technique;
4) Conducting precision machining to the section bar to prepare an organic-inorganic composite high-strength absorbable internal fixation device.

Preferably, the biomedical grade monomers include but are not limited to L-lactide, D(+)-lactide, DL-lactide, trimethylene carbonate, caprolactone, glycolide, or a mixture of the above monomers;

Preferably, number-average molar mass of the polylactic acid polymer is 100-600 kDa, and components of the polylactic acid polymer include but are not limited to poly-L-lactic acid, poly(D,L-lactide), poly-L-lactic acid-caprolactone copolymer (mass ratio of comonomers is 5:95-95:5) poly(D,L-lactide)-caprolactone copolymer (mass ratio of comonomers is 5:95-95:5), or a blend of the above polymers;

Preferably, main components of the bioactive nanoparticles are inorganic particles which can be used as a nucleating agent during polymer crystallization to refine crystalline grains, and are selected from one or a mixture of hydroxyapatite, tricalcium phosphate, calcium sulphate, calcium phosphate and magnesium sulphate;

Preferably, after surface modification, the dispersion condition of the nanoparticles can be improved and the biological activity of the nanoparticles can be reserved by initiating the polymerization of the biomedical grade monomers at an interface between the nanoparticles and the monomers.

Preferably, the step of lossless machining includes the processes of extrusion and granulation by a single screw rod or double screw rods, injection molding and annealing, wherein in the process of granulation, the temperature of the single screw rod or double screw rods is 40-200° C.; in the process of injection molding, the bucket temperature is 50-250° C., the mold temperature is 20-150° C., the injection pressure is 1-201 MPa, the injection time is 2-20 s, and the dwell time is 50-100 s; in the process of annealing, the cooling rate is 5-50° C./min, and the cooling time is 5-10 min;

Preferably, the autofrettage technique is a cold extrusion technique, the extrusion temperature is 60-200° C., the extrusion pressure is 100 N-100 kN, and the ratio of cross-sectional area before and after extrusion is 1-100;

Further, a multistage autofrettage method is to form the blank into the section bar through primary extrusion and enhancement, then extrude the section bar for several times, and cut to an appropriate size.

Specifically, the method comprises the following steps:
1) Synthesis: weighing polymer monomers, a catalyst and modified bioactive nanoparticles in a glove box, adding the mixture to a reactor, taking out the mixture after sealing, heating the mixture to 130-150° C. by oil bath, making the mixture react for 6 h under inert-gas protection, collecting a polymer by dichloromethane and absolute ethyl alcohol, and drying the polymer;
2) Granulation: using a single screw rod or double screw rods to conduct granulation at a temperature of 130-190° C. and a rotating speed of 4-80 rpm;
3) Injection molding: adding granules to a bucket at a bucket temperature of 50-250° C., a mold temperature of 20-150° C., an injection pressure of 1-20 MPa, an injection time of 2-20 s and a dwell time of 5-50 s;
4) Annealing: annealing the blank together with a mold at a cooling rate of 5-50° C./min and a cooling time of 5-10 min;
5) Cold extrusion: adding the blank obtained in step 4) into an extrusion bucket, and forming the blank into a section bar with a certain compression ratio by extrusion at a temperature below the melting temperature;
6) Molding: conducting precision machining to the section bar to obtain an absorbable internal fixation device with a specific shape.

According to the total weight of the degradable polymer (i.e., biomedical grade polymer) and the inorganic particles, the degradable polymer accounts for 20-99.999 wt % of the composite, and the inorganic particles account for 0.001-80 wt %.

The degradable polymer includes but is not limited to poly-L-lactic acid, poly(D,L-lactide), poly-L-lactic acid-caprolactone copolymer (mass ratio of comonomers is 5:95-95:5), poly(D,L-lactide)-caprolactone copolymer (mass ratio of comonomers is 5:95-95:5), or a blend of the above polymers.

The bioactive nanoparticles are inorganic particles which can be used as a nucleating agent during polymer crystallization to refine crystalline grains, and are selected from one or a mixture of hydroxyapatite, tricalcium phosphate, calcium sulphate, calcium phosphate and magnesium sulphate, with a particle diameter of 10-800 nm.

The step of lossless machining includes the processes of extrusion and granulation by a single screw rod or double screw rods, injection molding and annealing, wherein in the process of granulation, the temperature of the single screw rod or double screw rods is 40-200° C.; in the process of injection molding, the bucket temperature is 50-250° C., the mold temperature is 20-150° C., the injection pressure is 1-20 MPa, the injection time is 2-20 s, and the dwell time is 50-100 s; in the process of annealing, the cooling rate is 5-50° C./min, and the cooling time is 5-10 min.

The autofrettage technique is a cold extrusion technique, the extrusion temperature is 60200° C., the extrusion pressure is 100 N-100 kN, and the ratio of cross-sectional area before and after extrusion is 1-100; and the extrusion mold used in the present invention is designed according to the shape of the internal fixation device to be prepared.

Compared with the prior art, the present invention mainly has the following advantages:
(1) During machining, orientation of a polymer chain is strengthened due to the existence of an orientation force field, and directional growth of a crystalline region is enhanced by a crystallization enhancement process, so the comprehensive mechanical properties of the material is improved;
(2) Calcium- and phosphorus-based nanoparticles with osteoinductive activity are added, and an in situ polymerization method is used to improve the dispersion condition of the nanoparticles, so the biological activity of the composite is enhanced, the inflammatory reaction in a degradation process is reduced, crystalline grains are refined, and the mechanical properties of the material is enhanced;
(3) The machining is further processed according to the purpose of the material to adapt to the fixation of different bones and fracture surfaces, so the application scope of the material is expanded;
(4) The internal fixation device has excellent biological activity and mechanical properties, which not only has the function of biological fixation, but also has the function of inducing bone repair and regeneration.

The present invention is further described below in combination with the specific embodiments. It should be understood that the embodiments are only used for illustrating the present invention, not used for limiting the scope of the present invention. Experimental methods in which specific conditions are not specified in the following embodiments are carried out usually under conventional conditions or the conditions recommended by the manufacturer. Unless otherwise specified, the percentages and parts are based on weight.

Unless otherwise defined, all professional and scientific terms used herein have the same meanings as those familiar to those skilled in the art. In addition, any method and material similar or equal to the recorded content can be applied to the method of the present invention. The preferred implementation methods and materials described herein are for demonstration purposes only.

### Raw Material

| | Relevant physical and chemical parameters |
|---|---|
| Surface-modified | Silane coupling agent modified particles, |
| light-based apatite | particle size 5-500 nm, rod shape, L/D |
| | ratio 2-10 |
| Surface-modified | Low molecular weight polylactic acid modified |
| tricalcium phosphate | material with a particle size of 0.2-20 um |
| | (preferably 0.2-10 um) and a shape of shaped |
| | particles |

### General Test Method

### Mechanical Property

Flexural properties of the material are tested by a mechanical testing machine (CMT-2503, MTS) with a three-point bending method according to GB/T 9341-2008. The material is made into a rod with a diameter of 6 mm, a length of 40 mm for each specimen, a span of 20 mm, an experimental rate of 1 mm/min and a test temperature of 25° C.

The torsional properties of the device are determined by a small load torsional test system (55MT, Instron) according to ASTM F543-2013. The final device is taken as a specimen, with a loading speed of 1 rad/min and a test temperature of 25° C.

### Biological Activity

Hot pressing is conducted to the obtained composite by a press vulcanizer at 180° C. to make the composite into a thin membrane with a thickness of 0.2 mm; rBMSCs are used as model cells to evaluate the cytocompatibility of the composite material in vitro by MTT method; the morphology and adhesion condition of rBMSCs on the composite membrane are observed by SEM, and the differences in osteogenic differentiation abilities of rBMSCs on the composite membrane are evaluated through ALP staining and ALP activity experiments. In each of the above experiments, a pure polylactic acid membrane is used as a control specimen.

### Embodiment 1: Preparation of Internal Fixation Device

1) Synthesis: L-lactide, stannous octoate and modified hydroxyapatite (with an average particle diameter of 50 nm) are weighed in a glove box, added to a reactor, taken out after sealing, heated to 135° C. by oil bath, and made to react for 6 h under inert-gas protection; and a polymer is collected by dichloromethane and absolute ethyl alcohol, and dried;
2) Granulation: a single screw is used to conduct granulation at a temperature of 200° C. and a rotating speed of 40 rpm;
3) Injection molding: granules are added to a bucket at a bucket temperature of 170° C., a mold temperature of 30° C., an injection pressure of 10 MPa, an injection time of 3 s, a dwell pressure of 81MPa and a dwell time of 10 s;
4) Annealing: the blank is annealed together with a mold at a cooling rate of 30° C./min and a cooling time of 5 min;
5) Cold extrusion: the blank obtained in step 4) is added into an extrusion bucket, and the blank is formed into a section bar with a certain compression ratio by extrusion at a temperature of 160° C. and an extrusion pressure of 100 N;
6) Molding: turning machining is conducted to the section bar to obtain an absorbable internal fixation device.

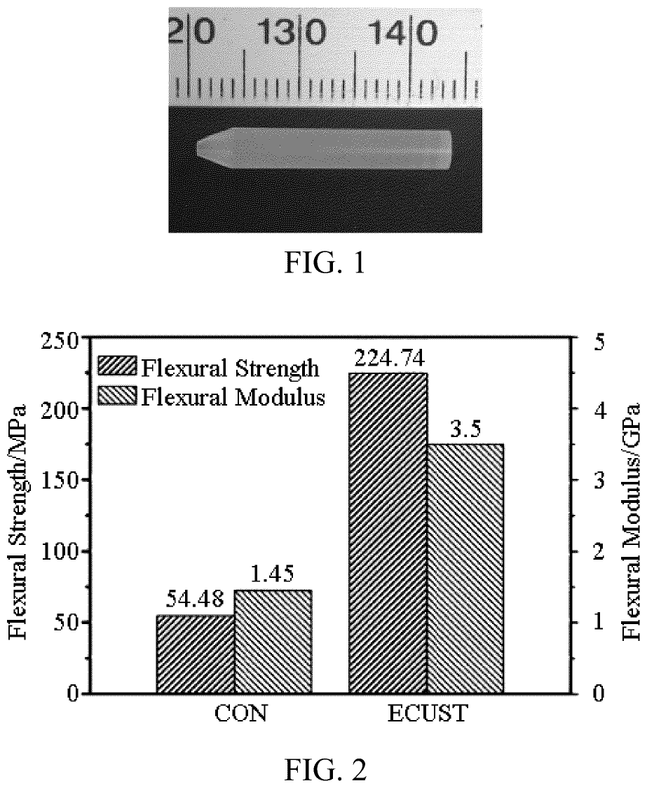

The internal fixation device obtained is shown in FIG. 1.

In the device, the mass fraction of L-lactide is 99.88 wt %, the mass fraction of the catalyst stannous octoate is 0.11 wt %, and the mass fraction of the modified hydroxyapatite is 0.01 wt %.

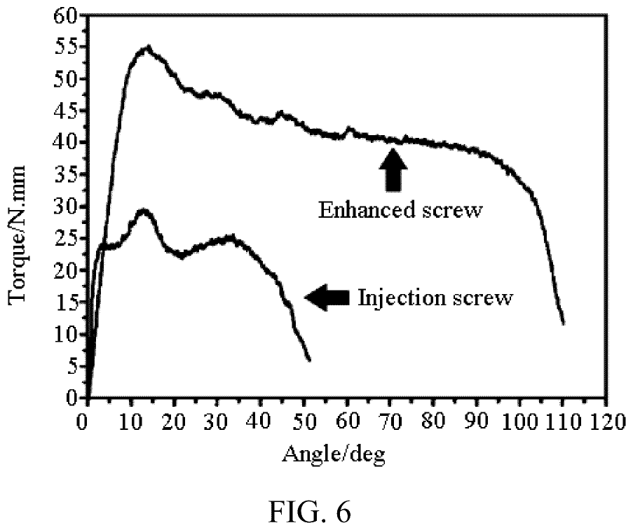

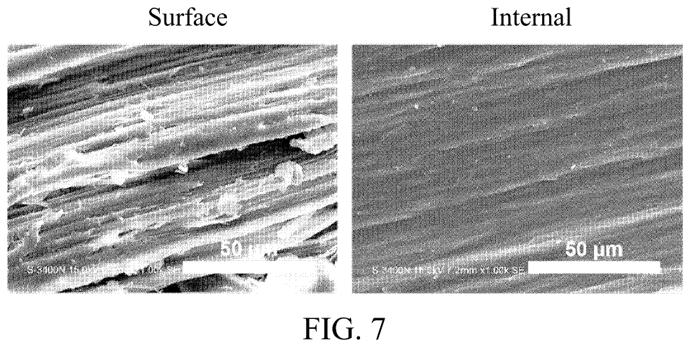

The differences in torsional properties of an enhanced internal fixation screw and the pure polylactic acid injection molding internal fixation device are shown in FIG. 6. A screw prepared by the above enhancement method has a peak torque of 55.11 N·mm, a peak angle of 115.5° and a flexural strength of 225 MPa. Compared with those of a screw obtained by direct injection molding, the peak torque of a cutting bone nail is increased by 87%, the peak angle is increased by 115%, and the flexural strength is increased by 3.13 times. The device prepared by enhancement is more robust than that prepared by injection molding.

### Embodiment 2: Determination of Mechanical Properties of Internal Fixation Device

A bar material obtained by cold extrusion in step 5) of embodiment 1 is used to test flexural properties of the material with a three-point bending method, with a length of 40 mm for each specimen, a span of 20 mm, an experimental rate of 1 mm/min and a test temperature of 25° C. A control specimen is a bar material obtained by melt extrusion using the same raw material after granulation in step 2), and the data is shown in FIG. 2. The bar enhanced by extrusion has a flexural strength of 224.74 MPa and a flexural modulus of 3.5 GPa, and the bar not enhanced has a flexural strength of 54.48 MPa and a flexural modulus of 1.45 GPa. Using the enhancement method described in this patent, the flexural strength can be increased by 3.13 times, and the flexural modulus can be increased by 1.41 times.

### Embodiment 3: Biological Activity of Internal Fixation Device

1) Synthesis: DL-lactide, stannous octoate and modified hydroxyapatite (with an average particle diameter of 100 nm) are weighed in a glove box, added to a reactor, taken out after sealing, heated to 135° C. by oil bath, and made to react for 6 h under inert-gas protection; and a polymer is collected by dichloromethane and absolute ethyl alcohol, and dried;
2) Granulation: a single screw is used to conduct granulation at a temperature of 190° C. and a rotating speed of 36 rpm;
3) Injection molding: granules are added to a bucket at a bucket temperature of 170° C., a mold temperature of 40° C., an injection pressure of 10 MPa, an injection time of 3 s, a dwell pressure of 81 MPa and a dwell time of 10 s;
4) Annealing: the blank is annealed together with a mold at a cooling rate of 20° C./min and a cooling time of 8 min;
5) Cold extrusion: the blank obtained in step 4) is added into an extrusion bucket, and the blank is formed into a section bar with a certain compression ratio by extrusion at a temperature of 140° C. and an extrusion pressure of 500 N;
6) Molding: turning machining is conducted to the section bar to obtain an absorbable internal fixation device.

In the device, the mass fraction of DL-lactide is 99.79 wt %, the mass fraction of modified hydroxyapatite (SHA) is 0.10 wt %, and the mass fraction of the catalyst is 0.11 wt %.

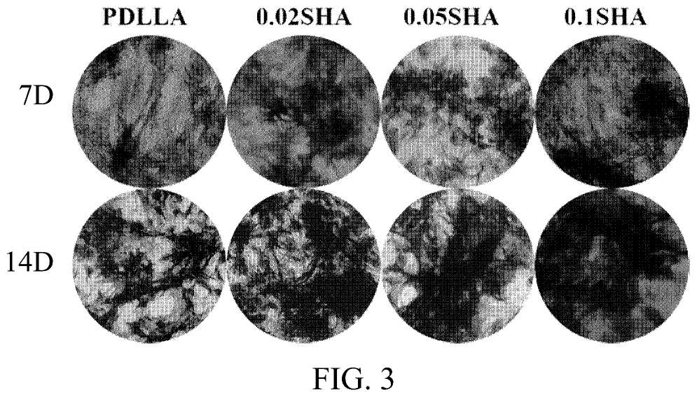

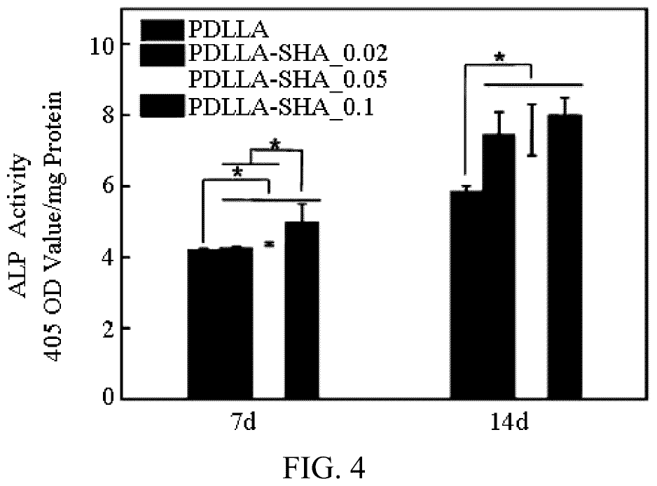

Hot pressing is conducted to the obtained composite by a press vulcanizer at 180° C. to make the composite into a thin membrane with a thickness of 0.2 mm; and the differences in osteogenic differentiation abilities of rat rBMSCs on the composite membrane are evaluated through ALP staining and ALP activity experiments. The results are shown in FIG. 3 and FIG. 4.

FIG. 3 shows ALP staining of rBMSCs co-cultured with a poly(D,L-lactide) (PDLLA) membrane and a poly(D,L-lactide)-modified hydroxyapatite composite (PDLLA-SHA_X) membrane for 7 days and 14 days. As can be seen from the figure, the ALP expression intensity of rBMSCs in the experimental group of PDLLA-SHA_0.1 at each time point is higher than that in other experimental groups, indicating that the PDLLA-SHA_0.1 experimental group has an optimal ability to promote osteogenic differentiation of rBMSCs.

FIG. 4 shows a quantitative analysis of ALP activity of rat rBMSCs on the polymer membranes for 7 days and 14 days. As can be seen from the figure, on the 7th day of co-culture of the cells and the materials, no significant difference is observed between the pure PDLLA membrane and the modified membranes of PDLLA-SHA_0.02 and PDLLA-SHA_0.05, and PDLLA-SHA_0.1 shows better ALP expression than the other three groups. After 14 days of co-culture, the PDLLA-SHA_0.02, PDLLA-SHA_0.05 and PDLLA-SHA_0.1 groups show significant differences from the pure PDLLA group, and the ALP activity of the PDLLA-SHA_0.1 group is still higher than that of the other three experimental groups, which is consistent with the condition of ALP staining. The above experiments show that the more SHA is introduced, the higher ALP activity is observed in the cells on the composite membrane. This is mainly because HA, as a main mineral component of natural bone, has excellent osseointegration and osteoconduction abilities.

### Reference Example 1: Preparation of Internal Fixation Device C1

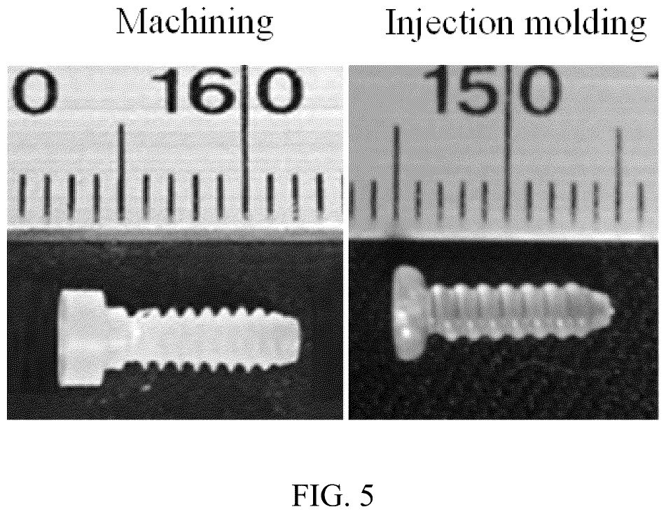

1) Synthesis: L-lactide and stannous octoate are weighed in a glove box, added to a reactor, taken out after sealing, heated to 135° C. by oil bath, and made to react for 6 h under inert-gas protection; and a polymer is collected by dichloromethane and absolute ethyl alcohol, and dried;
2) Granulation: a single screw is used to conduct granulation at a temperature of 190° C. and a rotating speed of 36 rpm;
3) Injection molding: granules are added to an injection molding machine to obtain a screw specimen by injection molding, as shown in FIG. 5.

The reference example is different from embodiment 1 in that: after synthesis and granulation, an internal fixation screw C1 is directly obtained by injection molding through the injection molding machine without being enhanced by cold extrusion. According to a torsional test of the device, the peak torque is 29.49 N·mm, and the peak angle is 51.40°, which are much smaller than those of an enhanced screw, as shown in FIG. 6. It is proved that the enhancement method proposed in the present invention can effectively improve the torsional strength of the specimen and overcome the shortcomings of high brittleness and easy torsional fracture caused by a simple polylactic acid injection molding process.

All references to the present invention are cited as references in the present application, just as each reference is cited as a reference separately. In addition, it should be understood that those skilled in the art could, after reading the above content of the present invention, implement various modifications to and variations of the present invention, and such equivalent forms also fall within the scope defined by appended claims of the present application.

1. A preparation method for an internal fixation device, comprising the following steps:
1) providing a first mixture, wherein the first mixture contains bioactive nanoparticles, a catalyst and biomedical grade monomers;
2) conducting polyreaction to the first mixture to obtain a first polymer;
3) conducting granulation to the first polymer;
4) injecting a granulation product into an injection molding machine to form a first blank by injection molding;
5) annealing the first blank to obtain a second blank;
6) conducting extrusion molding to the second blank, and conducting precision machining to the product obtained to prepare an internal fixation device.

2. The method according to claim 1, wherein the granulation has one or more characteristics selected from the following group:
1) the granulation is conducted using a single screw rod or double screw rods;
2) granulation temperature is 40-300° C.;
3) rotating speed of the single screw rod or double screw rods is 20-60 rpm.

3. The method according to claim 1, wherein the injection molding has one or more characteristics selected from the following group:
1) bucket temperature of the injection molding machine is 50-250° C.;
2) mold temperature is 10-150° C.;
3) injection time is 2-30 s;
4) injection pressure is 0.2-20 MPa;
5) dwell time is 5-100 s;
6) dwell pressure is 1-20 MPa.

4. The method according to claim 1, wherein the annealing has one or more characteristics selected from the following group:
1) the annealing is to anneal the first blank together with a mold;
2) cooling rate of the annealing is 5-50° C./min;
3) cooling time of the annealing is 4-15 min.

5. The method according to claim 1, wherein the extrusion molding has one or more characteristics selected from the following group:
1extrusion temperature of the extrusion molding is 60-200° C.;
2extrusion pressure of the extrusion molding is 50 N-100 kN.

6. The method according to claim 1, wherein the extrusion molding is repeated in step 6).

7. An internal fixation device, wherein the internal fixation device is prepared by the method of in claim 1.

8. The internal fixation device according to claim 7, wherein the internal fixation device has one or more characteristics selected from the following group:
1) flexural strength of the internal fixation device is 100-300 MPa;
2) flexural modulus of the internal fixation device is 2.5-5 GPa.

9. The internal fixation device according to claim 7, wherein the material forming the internal fixation device is selected from the following group: poly-L-lactic acid, poly(D,L-lactide), DL-lactide-caprolactone copolymer and L-lactide-glycolide copolymer.

10. The internal fixation device according to claim 7, wherein the internal fixation device is selected from the following group: an internal fixation compression screw, an interference screw, a rib rod and an internal fixation plate.
